(19) United States
(12) Patent Application Publication
(10) Pub. No.: US 2010/0069753 A1
(43) Pub. Date: Mar. 18, 2010

(54) Method and apparatus for detecting an acoustic obstruction

(76) Inventor: Russell James Fedewa, Shaker Heights, OH (US)

Correspondence Address: REED SMITH LLP, 2500 ONE LIBERTY PLACE, 1650 MARKET STREET, PHILADELPHIA, PA, 19103, US

(21) Appl. No.: 12/387,822

(22) Filed: May 6, 2009

Related U.S. Application Data
(60) Provisional application No. 61/050,749, filed on May 6, 2008.

(51) Int. Cl. A61B 8/14; A61N 7/00

(52) U.S. Cl. 600/439

(57) ABSTRACT

A method and apparatus is disclosed for determining the presence of an acoustic obstruction and for treating tissue comprising emitting ultrasound energy from an ultrasound transducer towards a targeted tissue region, sensing reflected ultrasound energy, determining whether an acoustic obstruction is present between the ultrasound transducer and the targeted tissue, and administering a HIFU Treatment if there is no acoustic obstruction present.

### CROSS REFERENCE

The present application claims priority under 35 U.S.C §119(e) to Provisional Application No. 61/050,749, filed May 6, 2008, the entire contents of which are incorporated herein by reference, including any references cited therein.

### FIELD OF THE INVENTION

The present invention relates to a method and apparatus for the non-invasive treatment of diseased tissue. In particular, the present invention relates to a method and apparatus for treatment of diseased tissue with High Intensity Focused Ultrasound (HIFU). The present invention also relates to a method and apparatus for detecting acoustic obstructions that may be present during the treatment of diseased tissue.

### BACKGROUND

Several techniques have been used in the past for the treatment of tissue including diseased tissue, such as cancer, to remove, destroy, or otherwise minimize the growth of the diseased tissue. One method of treating diseased tissue is surgical removal. Surgical removal, however, is invasive and can be time consuming and complex. Surgical treatment can result in serious complications, such as those from anesthesia. Thus, a more comprehensive and non-invasive treatment technique of similar or better efficacy is desired.

Another method of treating diseased prostate tissue is radiation. Radiation therapy, however, can destroy or damage nearby healthy tissue alongside the diseased tissue. Thus, a more refined and selective treatment technique is desired.

Another method of treating diseased prostate tissue is cryoablation. Cryoablation, however, can result in impotence. Thus, a treatment technique of similar or better efficacy without this harmful drawback is desired.

High Intensity Focused Ultrasound (HIFU) has been demonstrated to be a safe modality to treat diseased tissue noninvasively. For example, HIFU has been used to treat prostate cancer, kidney cancer, and testicular cancer. An exemplary system used to administer HIFU is the Sonablate® 500 (SB500) system available from Focus Surgery Inc., located at 3940 Pendleton Way, Indianapolis, Ind. 46226.

It is known that during the treatment of the prostate air bubbles between the probe and the rectal wall, such as between an acoustic membrane of the probe and the rectal wall, or calcification in the rectal wall may block the propagation of HIFU energy from being adequately delivered to the treatment site. Further, the application of HIFU energy in the presence of such an acoustic obstruction may result in damage to the rectal wall, such as a recto-urethral fistula. Traditionally, the physician is trained to observe multiple reverberations from an ultrasound image as an indication of an acoustic obstruction. A need exists for an automated method of detecting acoustic obstructions proximate to the rectal wall to avoid unwanted damage to the rectal wall.

Further exemplary embodiments of systems used to administer HIFU are disclosed in U.S. Patent Publication No. US2007/0010805 A1, filed Jul. 8, 2005, titled “Method and Apparatus for Treatment of Tissue;” U.S. Pat. No. 5,117,832; U.S. Patent Publication No. US2005/0240127, filed Mar. 2, 2005, titled “Ultrasound Phased Arrays;” U.S. Pat. No. 5,762,066; U.S. Pat. No. 5,036,855; U.S. Pat. No. 5,492,126; U.S. Pat. No. 6,685,640; and U.S. Pat. No. 5,993,389; (collectively “the HIFU Patents”), the disclosures of all of which are expressly incorporated herein by reference in their entireties.

### SUMMARY OF THE INVENTION

The present invention provides a method for treating tissue with HIFU Therapy. An ultrasound transducer emits ultrasound energy towards a targeted tissue region and senses ultrasound energy that is reflected. The ultrasound energy that is reflected is analyzed to determine whether or not there is an acoustic obstruction present. HIFU Treatment is administered to the tissue region if no acoustic obstruction is detected.

In an alternative embodiment, the present invention determines the presence of an acoustic obstruction by generating one or more line images from the sensed reflected ultrasound energy, determines a spectrum for the line images of the sensed reflected ultrasound energy, and analyzes the spectrum to determine the presence of an acoustic obstruction. In an example, an acoustic membrane is expanded to contact the targeted tissue, the tissue being a rectal wall of a patient. In another example, the step of determining the spectrum of the sensed reflected ultrasound energy includes the step of determining a Fourier Transform of the sensed reflected ultrasound energy. In a variation thereof the spectrum is normalized. In a further example, the steps of sending ultrasound energy from the transducer through the tissue component towards the treatment site and sensing reflected ultrasound energy with transducer as a function of time are repeated a plurality of times to form an image including a plurality of image lines. Prior to determining the spectrum of the sensed reflected ultrasound energy, the plurality of image lines are selected and a representative image line is determined. The representative image line is then used to determine the spectrum of the sensed ultrasound energy. In a variation thereof the representative image line is an average of the plurality of selected image lines. In yet a further example, the spectrum is determined through an autoregressive filter. In still another example, if the acoustic obstruction is present, a HIFU Treatment at the treatment site is prevented. In yet still another example, if the acoustic obstruction is absent, a HIFU Treatment at the treatment site is allowed.

In another embodiment, the present invention provides a treatment apparatus for treating tissue comprising a probe including an acoustic membrane covering at least a portion of the probe and an ultrasound transducer positioned behind the acoustic membrane. The ultrasound transducer is configured to emit ultrasound energy and to sense ultrasound energy. The controller, as described below, performs certain signal processing functions on the reflected signal. This can be done by the controller itself or by the controller in conjunction with a separate processing device. The controller is configured to operate the transducer in an imaging mode and in a therapy mode wherein a plurality of treatment sites are treated with a HIFU Therapy with the transducer. The controller is configured to detect the presence of an acoustic obstruction by determining a spectrum of at least one image and comparing a value of the spectrum to a threshold value. If the value exceeds the threshold then an acoustic obstruction is present. In an example, the controller is further configured to prevent operation of the transducer in therapy mode based on a detection of an acoustic obstruction. In another example, the value is compared to a threshold value and an acoustic obstruction is detected if the value exceeds the threshold value.

Although the techniques, methods, and apparatus discussed herein have applicability to the treatment of tissue in general, this discussion will focus primarily on the treatment of prostate tissue including Benign Prostatic Hyperplasia (BPH) and prostatic cancer. However, the disclosed apparatus and methods will find applications in localization and treatment of a wide range of diseases which manifest themselves in a localized or “focal” manner, including cancers of the breast, brain, liver, and kidney. As explained herein, the disclosed apparatus uses an intracavity probe which will be particularly useful for focal diseases which are accessible to a transesophageal, laparoscopic or transvaginal probe. Such diseases include esophageal cancer, cancer in the trachea and urethra, ulcers in the stomach and duodenum, and pancreatic cancer. Moreover, a transvaginal probe according to the present invention will provide a minimally invasive sterilization procedure on an outpatient basis, as well as therapy for fibroids, and endometrial ablation. Additionally, in the case of a transducer with multiple focal lengths, blood vessels may be selectively targeted to effect coagulation and cauterization of internal bleeding.

As used herein the term “HIFU Therapy” is defined as the provision of high intensity focused ultrasound (“HIFU”) to a portion of tissue at or proximate to a focus of a transducer. It should be understood that the transducer may have multiple foci and that HIFU Therapy is not limited to a single focus transducer, a single transducer type, or a single ultrasound frequency. As used herein the term “HIFU Treatment” is defined as the collection of one or more HIFU Therapies. A HIFU Treatment may be all of the HIFU Therapies administered or to be administered, or it may be a subset of the HIFU Therapies administered or to be administered. As used herein the term “HIFU System” is defined as a system that is at least capable of providing a HIFU Therapy.

Additional features of the present invention will become apparent to those skilled in the art upon consideration of the following detailed description of the illustrative embodiments exemplifying the best mode of carrying out the invention as presently perceived.

### BRIEF DESCRIPTION OF THE DRAWINGS

The detailed description of the drawings particularly refers to the accompanying figures in which:

FIG. 1 is schematic view of an exemplary HIFU System of the present invention, the HIFU System being capable of imaging the tissue of the patient and to provide HIFU Therapy to at least a portion of the tissue at or proximate to a focus of a transducer of the HIFU System.

FIG. 2 is an exemplary embodiment of the HIFU System of FIG. 1.

FIG. 3 is an exemplary image contrasting the received response for the absence of an acoustic obstruction and the presence of an acoustic obstruction.

FIG. 4 is a graph showing an exemplary line image of a portion of a treatment region including the rectal wall and prostate and not including an acoustic obstruction proximate to the transducer and an exemplary line image of a portion of a treatment region including the rectal wall and prostate and including an acoustic obstruction proximate to the transducer.

FIG. 5 is a graph showing an exemplary line image of a portion of a treatment region including the rectal wall and prostate and not including an acoustic obstruction proximate to the transducer and an exemplary line image of a portion of a treatment region including the rectal wall and prostate and including an acoustic obstruction proximate to the transducer.

FIG. 6A is a representation of the rectum of the patient along with the probe, the transducer, and an acoustic membrane covering a portion of the probe and the HIFU System of FIG. 1.

FIG. 6B is a representation of the view of FIG. 6A wherein an acoustic obstruction is positioned proximate to the transducer, illustratively between the acoustic membrane and the rectal wall of the rectum.

### DETAILED DESCRIPTION OF THE DRAWINGS

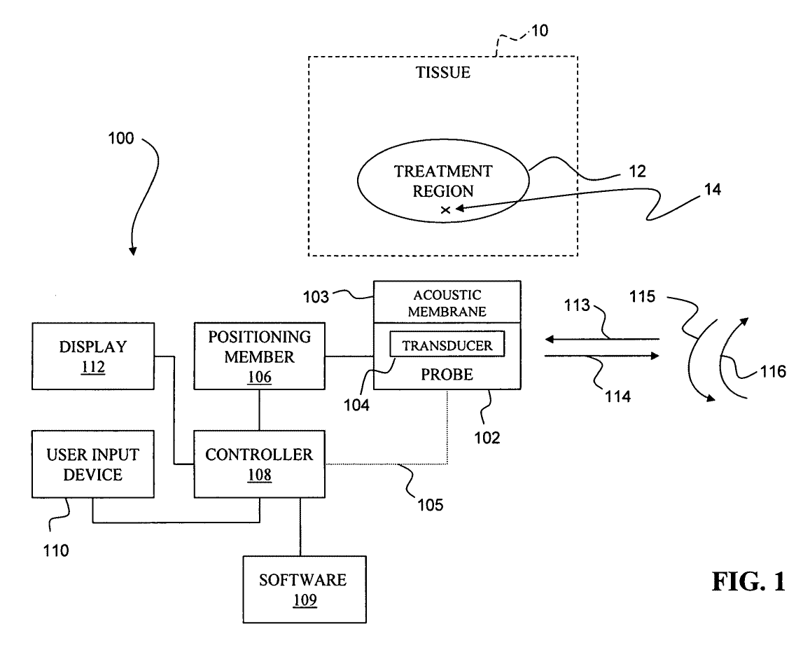

An exemplary HIFU System 100 is shown in FIG. 1. HIFU System 100 includes a probe 102 having a transducer member 104, a positioning member 106, a controller 108 operably coupled to probe 102 and the positioning member 106, a user input device 110 (such as keyboard, trackball, mouse, and/or touch screen), and a display 112. Probe 102 is operably connected to controller 108 through positioning member 106. However, as indicated by line 105 probe 102 may be directly connected with controller 108. Positioning member 106 is configured to linearly position transducer member 104 along directions 113, 114 and to angularly position transducer member 104 in directions 115, 116.

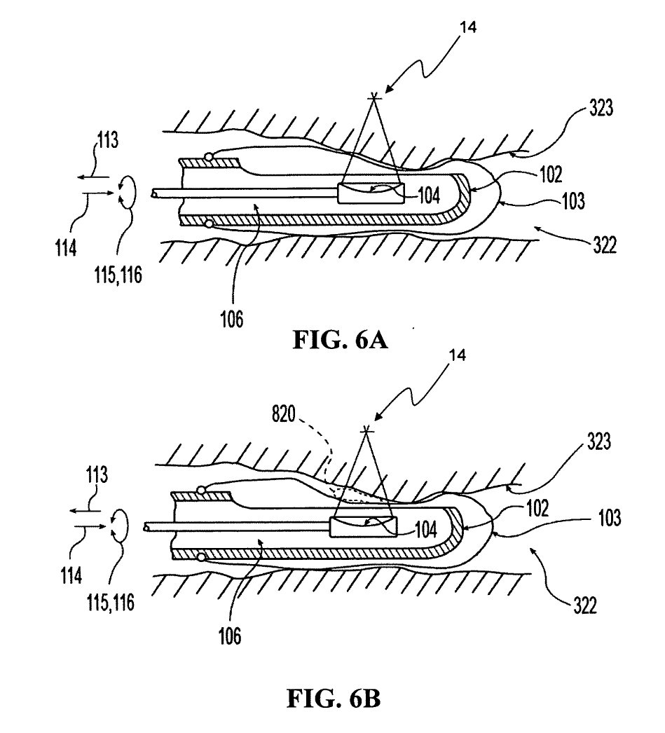

Transducer member 104 is positioned generally proximate to a region of tissue 10. In the case of the prostate, transducer 104 is positioned generally proximate to the prostate by the transrectal insertion of probe 102. An example is shown in FIG. 6A wherein transducer member 104 is positioned proximate to a rectal wall 323 of a patient.

Transducer member 104 is moved by positioning member 106 and controlled by controller 108 to provide imaging of at least a portion of tissue 10 including at least one treatment region 12 and to provide HIFU Therapy to portions of the tissue within at least one treatment region 12.

HIFU System 100 may operate in an imaging mode wherein at least a portion of tissue 10 may be imaged and in a therapy mode wherein HIFU Therapy is provided to portions of tissue 10 within at least one treatment region, such as a treatment site 14. Treatment region 12 is defined as one or more portions of tissue which are to be treated during a HIFU Treatment. Treatment region 12 is illustratively shown as a continuous region. However, a treatment region might involve two or more distinct regions. In one example, as described in U.S. Patent Publication No. US2007/0010805 A1, filed Jul. 8, 2005, the disclosure of which is expressly incorporated by reference, treatment region 12 includes a plurality of treatment sub-portions, such as treatment segments.

In one embodiment, transducer member 104 is a single crystal two element transducer. An exemplary transducer is disclosed in U.S. Pat. No. 5,117,832, the disclosure of which is expressly incorporated herein by reference. In a preferred embodiment, transducer 104 is capable of providing imaging of at least a portion of tissue 10 and of providing HIFU Therapy to at least a portion of tissue 10 within treatment region 12.

However, the present invention is not limited to the type of transducer implemented. On the contrary, various transducer geometries having a single focus or multiple foci and associated controls may be used including transducers which are phased arrays, such as the transducers disclosed in U.S. Patent Publication No. US2005/0240127 A1, filed Mar. 2, 2005, titled “Ultrasound Phased Arrays,” the disclosure of which is expressly incorporated herein by reference. Additional exemplary transducers and associated controls are disclosed in U.S. Pat. No. 5,762,066; U.S. Pat. No. 5,036,855; U.S. Pat. No. 5,492,126; U.S. Pat. No. 6,685,640, each of which is expressly incorporated herein by reference.

In one embodiment, a portion of probe 102 is covered by an acoustic membrane 103. Acoustic membrane 103 is an expandable membrane whose overall size is increased by placing a fluid in an interior of acoustic membrane 103. In one embodiment, the fluid is water or a generally acoustic transparent material and is provided by a reservoir or a chiller. The fluid may be used to remove heat from proximate to transducer 104 as well as expanding acoustic membrane 103. In one embodiment, acoustic membrane 103 is expanded such that it contacts or generally is adjacent to the surrounding tissue, such as a tissue component like rectal wall 323, as shown in FIG. 6A. In one embodiment, acoustic membrane 103 is a condom placed over a tip of probe 102, sealed with o-rings, and filled with water. Exemplary acoustic membranes and details of their operation in relation to respective other portions of exemplary HIFU Systems are provided in U.S. Pat. No. 5,762,066, and U.S. Pat. No. 5,993,389, the disclosures each of which are expressly incorporated by reference herein.

In one embodiment, controller 108 is configured to execute one or more of the methods discussed herein. In one embodiment, at least a portion of each method executed by controller 108 is provided as a portion of software 109. In one embodiment, the HIFU Systems disclosed in U.S. Patent Publication No. US2007/0010805 A1, filed Jul. 8, 2005, titled “Method and Apparatus for Treatment of Tissue” may be modified to incorporate the teaching of the present disclosure. The disclosure of U.S. Patent Publication No. US2007/0010805 A1 is expressly incorporated by reference herein.

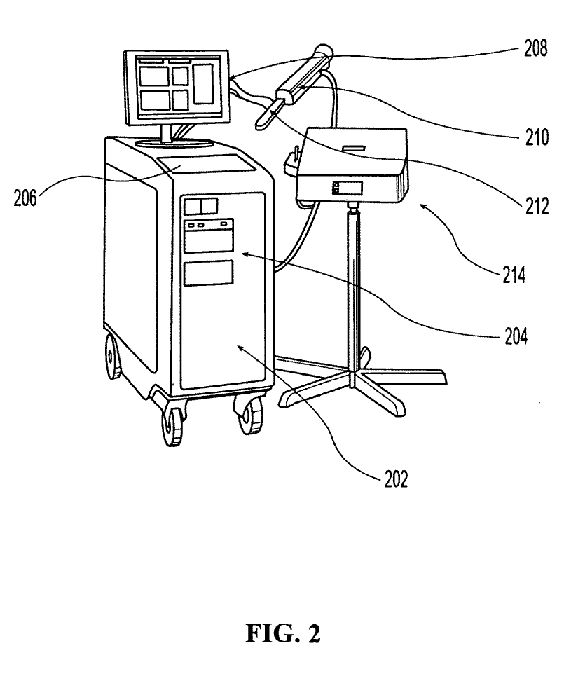

Referring to FIG. 2, an exemplary HIFU System 200 is shown, the Sonablate® 500 HIFU System available from Focus Surgery, Inc., located at 3940 Pendleton Way, Indianapolis, Ind. 46226. HIFU System 200 includes a console 202 which houses or supports a controller (not shown), such as a controller and associated software; a printer 204 which provides hard copy images of tissue 10 and/or reports; a user input device 206 such as a keyboard, trackball, and/or mouse; and a display 208 for displaying images of tissue 10 and software options to a user, such as a color display. Further, shown is a probe 210 which includes a transducer member (not shown), and a positioning member (not shown). Also shown is an articulated probe arm 212 which is coupled to console 202. Articulated probe arm 212 orients and supports probe 210. A chiller 214 is also shown. Chiller 214, in one embodiment, provides a water bath with a heat exchanger for the transducer member of probe 210 to actively remove heat from the transducer member during a HIFU Treatment.

In one embodiment, HIFU System 100 is configured to detect the presence of an acoustic obstruction in the propagation path of the HIFU energy which are not generated as a result of the HIFU Treatment (not hyperechoic features). In the case of treating the prostate, exemplary acoustic obstructions 820 (see FIG. 6B) include air bubbles between probe 102 and rectal wall 323 or calcifications in the rectal wall itself. The presence of an acoustic obstruction blocks or at least severely limits the amount of HIFU energy that may proceed to the proposed treatment site 14 during a HIFU Therapy. Similarly, the presence of an acoustic obstruction blocks or severely limits the amount of ultrasound energy that may penetrate beyond the obstruction to image the tissue behind the obstruction. As such, the acoustic obstruction may both limit the imaging ability of HIFU System 100 and limit the effectiveness of HIFU Therapy provided by HIFU System 100. Further, in the case of HIFU Therapy tissue proximate to the obstruction may be unintentionally damaged by the application of HIFU energy, such as rectal wall 323.

As is known, in ultrasound imaging an acoustic signal is transmitted into a medium and portions of the ultrasound signal are reflected back from portions of the medium and received by a transducer. The magnitude of these reflections is due to the properties of the portions of the medium causing the reflection. In the case of an acoustic obstruction proximate to the transducer, the acoustic signal is largely reflected by the acoustic obstruction back to the transducer. A portion of this large reflected signal is reflected by the transducer back into the medium wherein it is again reflected by the acoustic obstruction. This bouncing of the acoustic signal back-and-forth between the acoustic obstruction and the transducer generates a generally periodic acoustic signal in time at intervals corresponding to the distance between the transducer and the obstruction. This repetitive pattern may be used to detect the presence of the acoustic obstruction.

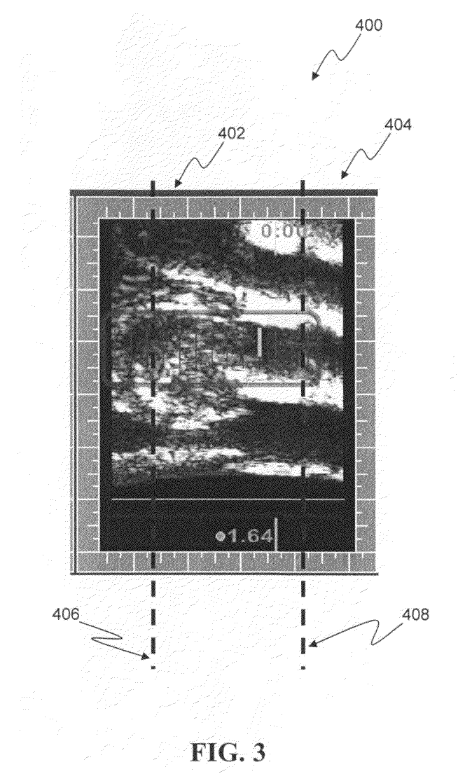

Referring to FIG. 3, an ultrasound image 400 is shown. Once the ultrasound image has been obtained, a portion or sub-image is extracted from the image which generally corresponds to either a proposed treatment site or a treatment site that has just been treated with HIFU energy. In one embodiment, the image and the sub-image correspond to an on-axis configuration between the transducer and the given treatment site. The sub-image is chosen to correspond to lines or columns of pixels that generally correspond to the extent of the treatment site. The intensity values of the corresponding pixels in each line or column are averaged to produce a line image which provides the averaged intensity value as a function of time. Image 400 includes a first region 402 wherein no acoustic obstruction is located at the interface of acoustic membrane 103 with the surrounding tissue and a second region 404 wherein an acoustic obstruction is located at the interface of acoustic membrane 103. A line 406 and a line 408 are shown to represent one or more image lines of image 400 in region 402 and region 404, respectively. Lines 406 and 408 may represent a single line image or representative line image of a plurality of line images. In one embodiment, the line images are generated by processing the sensed reflected ultrasound energy. In another embodiment, a representative line image is an average of a plurality of line images.

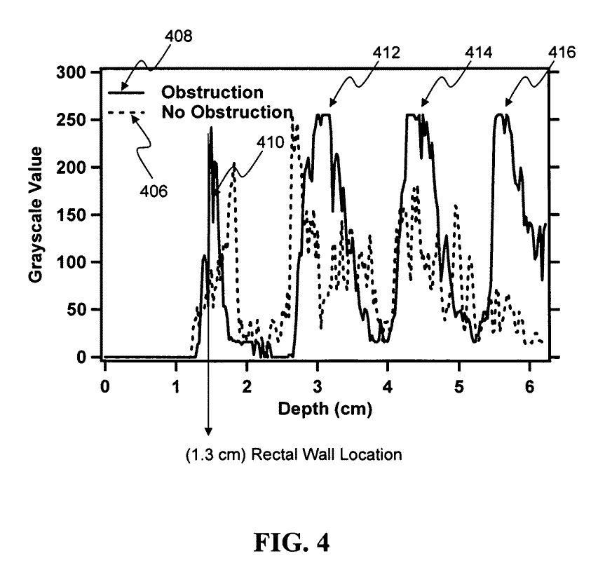

Referring to FIG. 4, the grayscale values of line images 406 and 408 are represented. A depth of 0 cm corresponds to the bottom of the image 400 in FIG. 3. As may be seen in FIG. 4, line image 406 does not exhibit a strong repetitive pattern. Line image 408 does exhibit a strong repetitive pattern, namely regions 410, 412, 414, and 416. As noted in FIG. 4, rectal wall 323 is located at 1.3 cm from transducer 104. As discussed in U.S. Publication No. US2007/0010805 A1, filed Jul. 8, 2005, the disclosure of which is expressly incorporated by reference herein, the spacing of these regions may be used to detect the presence of an acoustic obstruction 820 at the interface between acoustic membrane 103 and rectal wall 323.

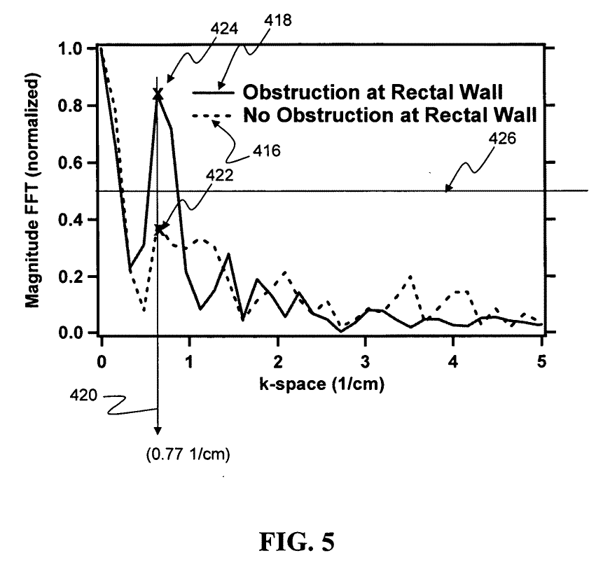

Referring to FIG. 5, another method of detecting the presence of acoustic obstruction 820 is illustrated. In FIG. 5, two line images 416 and 418 are shown. Line image 416 is related to line image 406 and line image 418 is related to line image 408. Line images 416 and 418 illustrate a spectrum for line images 406 and 408, respectively. In the illustrated example, this spectrum is a Fourier Transform the respective line image 406 and 408 which has been normalized. The Fourier Transform may be determined by a controller through a Fast Fourier Transform program.

The spectrum is analyzed to determine the presence or absence of acoustic obstruction 820. The abscissa axis in FIG. 5 is known as the k-space and corresponds to 1/s (in time) or 1/cm (in distance). As stated in regard to FIG. 4, the location of rectal wall 323 is 1.3 cm. As such, the k-space value corresponding to rectal wall 323 is 0.77 (1/cm).

As shown in FIG. 5, line image 416 corresponding to no acoustic obstruction has a value 422 which is below a threshold value of 0.5 represented by line 426. Although line 426 is shown as corresponding to a value of 0.5, it should be understood that line 426 and hence the threshold value may be higher or lower than 0.5. Line image 418 corresponding to an acoustic obstruction 820 being located between acoustic membrane 103 and rectal wall 323 has a value 424 which is above the threshold value represented by line 426.

A controller, when analyzing line image 416, compares value 422 to the threshold value. Since value 422 is below the threshold value, the controller determines that no acoustic obstruction is present. As such, HIFU Therapy may be carried out. The controller, when analyzing line image 418, compares value 424 to the threshold value. Since value 424 is at or above the threshold value, the controller determines that acoustic obstruction 820 is present. As such, HIFU Therapy may not be carried out. In response, HIFU System 100 may pause imaging or treatment due to the presence of an acoustic obstruction, as represented by block 866.

At this point HIFU System 100 may either simply wait a predetermined time followed by an attempt to re-image the portion of treatment region 12. Some types of acoustic obstructions, such as air bubbles introduced during the insertion of probe 102 or generated by patient flatulence, are transient acoustic obstructions. Other types of acoustic obstructions, such as calcification in the rectal wall 323, are generally permanent acoustic obstructions.

In the case of transient acoustic obstructions, the acoustic obstruction may migrate away from the probe 102 or may be removed by moving the probe 102 or by direct physician intervention. In the case of permanent acoustic obstructions, either the patient is not considered a good candidate for HIFU Treatment or the obstructed portion of treatment region 12 is simply not treated with HIFU Therapy. In one embodiment, wherein a phased array transducer is used, the obstructed portion of treatment region 12 may be treated by translating the transducer or activating a spaced apart aperture of the transducer and treating the obstructed portion from an off-axis position. An exemplary phased array transducer is provided in U.S. Patent Publication No. US2005/0240127 A1, filed Mar. 2, 2005, the disclosure of which is expressly incorporated by reference herein.

In one embodiment, the acoustic obstruction method includes the steps of extracting from an image a set of line images. The extracted set of line images are then averaged together. The spectral characteristics of the averaged line image are determined. In one example, a Fourier Transform is performed on the averaged line image. In one example, an autoregressive filter is used to determine the spectral characteristics of the averaged line image. The spectrum of the line image is analyzed at a k-space value corresponding to the inverse of the rectal wall position. The value of the spectrum at the k-space value corresponding to the inverse of the rectal wall position is then compared to a threshold value. If the value of the spectrum value at that position exceeds the threshold value, an acoustic obstruction is present. The HIFU System is prevented from providing HIFU Therapy until the acoustic obstruction is no longer detected.

The acoustic detection methods described herein are based on or otherwise utilize the intensity values of one or more pixels in one or more images to detect acoustic features, classify acoustic features, and/or to make one or more treatment decisions. The intensity values of the pixels in the one or more images are based on the electrical radio frequency signals generated by the transducer in response to detected acoustic energy. As such, in one embodiment, the herein described methods may be based on the radio frequency signals themselves or various conditioned forms thereof instead of the intensity values of image pixels.

Although the invention has been described in detail with reference to certain illustrated embodiments, variations and modifications exist within the spirit and scope of the invention as described and defined in the following claims.

1. A method of treating tissue located at a treatment site, the method including the steps of:
emitting ultrasound energy from an ultrasound transducer towards a targeted tissue region;
sensing reflected ultrasound energy;
determining whether an acoustic obstruction is present between the ultrasound transducer and the treatment site; and
administering a HIFU Treatment if there is no acoustic obstruction present,
wherein presence of an acoustic obstruction is determined by generating one or more line images from the sensed reflected ultrasound energy, determining a spectrum for the line images of the sensed reflected ultrasound energy, and analyzing the spectrum to determine the presence of an acoustic obstruction.

2. The method of claim 1 wherein the line images are generated from pixel intensity values of an ultrasound image.

3. The method of claim 2, wherein the pixel intensity values are grayscale values.

4. The method of claim 1, wherein the step of analyzing the spectrum to determine the presence of an acoustic obstruction comprises the step of comparing a selected spectrum value to a preselected threshold value.

5. The method of claim 4, wherein the selected spectrum value corresponds to a k-space value related to a distance from the transducer to the targeted tissue region.

6. The method of claim 4, wherein a controller determines that an acoustic obstruction is not present if the selected spectrum value is less than the preselected threshold value.

7. The method of claim 4, wherein a controller determines that an acoustic obstruction is present if the selected spectrum value is greater than or equal to the preselected threshold value.

8. The method of claim 1, wherein the spectrum is a Fourier Transform of the line images.

9. The method of claim 8, wherein the Fourier Transform is determined by a Fast Fourier Transform calculation.

10. The method of claim 8, wherein the Fourier Transform is normalized.

11. The method of claim 1, wherein the spectrum is determined by an autoregressive filter.

12. The method of claim 1, wherein the one or more line images is determined based on electrical radio frequency signals generated by the ultrasound transducer in response to the sensed reflected ultrasound energy.

13. The method of claim 1, further comprising the step of determining a spectrum for the one or more line images by averaging the line images.

14. The method of claim 13 wherein a Fourier Transform is performed on the averaged line image.

15. The method of claim 14, wherein the Fourier Transform is determined by a Fast Fourier Transform calculation.

16. The method of claim 13, wherein the spectrum is determined by an autoregressive filter.

17. A method for detecting the presence of an acoustic obstruction in tissue to be treated with HIFU, the method comprising the steps of:
sensing reflected ultrasound energy; and
determining whether an acoustic obstruction is present between the ultrasound transducer and the treatment site,
wherein presence of an acoustic obstruction is determined by generating one or more line images from the sensed reflected ultrasound energy, determining a spectrum for the line images of the sensed reflected ultrasound energy, and analyzing the spectrum to determine the presence of an acoustic obstruction.

18. The method of claim 17 wherein the line images are generated from pixel intensity values of an ultrasound image.

19. The method of claim 18, wherein the pixel intensity values are grayscale values.

20. The method of claim 17, wherein the step of analyzing the spectrum to determine the presence of an acoustic obstruction comprises the step of comparing a selected spectrum value to a preselected threshold value.

21. The method of claim 20, wherein the selected spectrum value corresponds to a k-space value related to a distance from the transducer to the targeted tissue region.

22. The method of claim 20, wherein a controller determines that an acoustic obstruction is not present if the selected spectrum value is less than the preselected threshold value.

23. The method of claim 20, wherein a controller determines that an acoustic obstruction is present if the selected spectrum value is greater than or equal to the preselected threshold value.

24. The method of claim 17, wherein the spectrum is a Fourier Transform of the line images.

25. The method of claim 24, wherein the Fourier Transform is determined by a Fast Fourier Transform calculation.

26. The method of claim 24, wherein the Fourier Transform is normalized.

27. The method of claim 17, wherein the spectrum is determined by an autoregressive filter.

28. The method of claim 17, wherein the one or more line images is determined based on electrical radio frequency signals generated by the ultrasound transducer in response to the sensed reflected ultrasound energy.

29. The method of claim 17, further comprising the step of determining a spectrum for the one or more line images by averaging the line images.

30. The method of claim 29 wherein a Fourier Transform is performed on the averaged line image.

31. The method of claim 30, wherein the Fourier Transform is determined by a Fast Fourier Transform calculation.

32. The method of claim 29, wherein the spectrum is determined by an autoregressive filter.

33. A treatment apparatus for treating tissue, the apparatus comprising:
a probe including an acoustic membrane covering at least a portion of the probe, an ultrasound transducer positioned behind the acoustic membrane, the transducer being configured to emit ultrasound energy to a treatment tissue and to sense ultrasound energy; and
a controller coupled to the transducer, the controller being configured to operate the transducer in an imaging mode and in a therapy mode, the controller being capable of detecting an acoustic obstruction between the transducer and the treatment tissue,
wherein the controller generates one or more line images from sensed ultrasound energy, determines a spectrum for line images of the sensed ultrasound energy, and analyzes the spectrum to determine a presence of an acoustic obstruction.

34. The apparatus of claim 33, wherein the controller operates in the imaging mode wherein at least one image of the treatment tissue is obtained from ultrasound energy sensed by the transducer.

35. The apparatus of claim 33, wherein the controller operates in the therapy mode wherein a plurality of treatment sites are treated with HIFU Therapy with the transducer.

36. The apparatus of claim 33, wherein the controller is capable of detecting the acoustic obstruction by comparing a selected spectrum value to a preselected threshold value.

37. The apparatus of claim 33, wherein the line images are generated from pixel intensity values of an ultrasound image.

38. The apparatus of claim 37, wherein the pixel intensity values are grayscale values.

39. The apparatus of claim 33, wherein the line images is determined based on electrical radio frequency signals generated by the ultrasound transducer in response to the sensed reflected ultrasound energy.

40. The apparatus of claim 36, wherein the selected spectrum value corresponds to a k-space value related to a distance from the transducer to the targeted tissue region.

41. The apparatus of claim 36, wherein the controller determines the spectrum of the one or more line images by performing a Fourier Transform calculation.

42. The apparatus of claim 41, wherein the Fourier Transform is determined by a Fast Fourier Transform calculation.

43. The apparatus of claim 33, wherein the controller determines the spectrum of the one or more line images by an autoregressive filter.

44. The apparatus of claim 36, wherein the controller detects the presence of an acoustic obstruction when the selected spectrum value is greater than or equal to the preselected threshold value.

45. The apparatus of claim 36, wherein the controller does not detect the presence of an acoustic obstruction when the selected spectrum value is less than the preselected threshold value.
